(19) United States
(12) Patent Application Publication
(10) Pub. No.: US 2007/0161884 A1
(43) Pub. Date: Jul. 12, 2007

(54) Methods, systems, and computer program products for providing dynamic data of positional localization of target implants

(75) Inventors: Robert D. Black, Raleigh, NC (US); Gregory Glenwood Mann, Raleigh, NC (US); Steven R. Widener, Wake Forest, NC (US)

Correspondence Address: MYERS BIGEL SIBLEY & SAJOVEC, PO BOX 37428, RALEIGH, NC, 27627, US

(73) Assignee: SICEL TECHNOLOGIES, INC., Morrisville, NC (US)

(21) Appl. No.: 10/551,366

(22) PCT Filed: Mar. 29, 2004
(86) PCT No.: PCT/US04/09483

§ 371 Date: Aug. 29, 2006

Related U.S. Application Data
(60) Provisional application No. 60/459,697, filed on Apr. 2, 2003.

(51) Int. Cl. A61B 5/05

(52) U.S. Cl. 600/407

(57) ABSTRACT

Systems for locating implanted in vivo sensors systems adapted for use with an external beam radiation therapy delivery source include: (a) an external solenoid member; (b) an articulated arm opertively associated with the external solenoid member, wherein, in operation, the articulated arm is configured to translate the solenoid; (c) a controller configured to direct the movement of the articulated arm, the controller being in communication with the power source configured to power the external solenoid; (d) at least one implantable sensors unit, wherein the at least one implantable sensor unit is configured to sense at least one predetermined parameter of interest in vivo, and wherein the at least one implantable sensor unit comprises a solenoid, and wherein, in operation, the sensor unit solenoid corporates with the external solenoid to generate a magnetic coupling signal having a signal strength that varies based on the possession of the external solenoid member relative to the implanted sensors unit; (e) a computer module in communication with the controller comprising computer programmed code that evaluates the coupling signal strength in relation to the position of the external solenoid and determines the position of the at least one sensor unit; and (f) an external reader configured to wirelessly communicate with the at least one implantable sensor unit to obtain data associated with the at least one predetermined parameter of interest.

### RELATED APPLICATIONS

This application claims the benefit of priority of U.S. Provisional Patent Application No. 60/459,697, filed Apr. 2, 2003, the contents of which are hereby incorporated by reference as if recited in fill herein.

### FIELD OF THE INVENTION

This invention relates to systems for determining the location of implanted devices.

### BACKGROUND OF THE INVENTION

Radiation therapy is used to treat localized cancers or other conditions. Examples of radiation therapy treatments include conventional external beam radiation therapy, as well as three-dimensional conformal external beam radiation, intensity modulated radiation therapy (IMRT), a “gamma knife” that employs a highly focused gamma ray radiation obtained from crossing or collimating several radiation beams, stereotactic radiosurgery and brachytherapy.

The efficacy of the radiation treatment can depend on the total dose of radiation delivered to the target region. However, the amount of radiation effectively delivered to the target region (as well as the amount delivered to healthy tissue) can vary from a desired or planned amount. The variation can be particularly problematic when radiation therapy is used on deep tumors, when the therapy is delivered to tumors located close to healthy sensitive regions or organs, and/or when complex beam signals are employed.

Typically, the radiation therapy is directed not only to the known tumor location, but also to healthy tissue proximate the tumor based on a treatment margin. The more imprecise the radiation delivery is thought to be, the larger the planned treatment margin that may be used. As radiation can be detrimental to healthy tissue, a therapy goal should be to use smaller treatment margins while delivering radiation doses in the planned amounts and to the planned location. However, delivering external beam radiation doses in the desired dose amount to the actual tumor site can be complicated as the tumor and/or markers used to locate and guide the radiation therapy may shift over time, either during or between radiation sessions.

For example, tumor motion can occur during the active delivery of the radiation due to normal biophysical actions. That is, in certain locations in the body, such as in the prostate, movement of target tissue may occur during radiation treatment, primarily attributable to the patient's breathing or filling and/or voiding of the bladder. Thus, dynamic changes in the position of the tumor during active radiation delivery can increase the potential of collateral damage to healthy or non-targeted tissue.

In the past, systems using positional markers have been proposed to determine spatial positioning information for targets from within a patient's body. Such localization may be used to direct or guide the radiation therapies. For example, WO 02/100485 to Mate et al. proposes using an external sensor array having a plurality of sensors, the sensors having known geometry relative to each other, and a beacon that is transmitted from a location that is in or on the body, typically near the target. The beacon can be generated from an implanted beacon with a ferrite core wrapped by a conductive winding. As described, the system then defines a target isocenter using the beacon signal and the sensors in the external array and compares the location of the target isocenter with the machine isocenter before and during radiation therapy.

U.S. Pat. No. 6,239,724 to Doron et al. proposes a system with an implantable telemetry unit that employs three transducers: one for powering the implanted unit upon receipt of an externally generated signal; one for receiving an externally generated positioning field signal; and one for transmitting a locating signal in response to the receipt of the positioning field signal. The system communicates with the implanted telemetry unit using coupling techniques, such as ultrasound coupling to communicate with the unit(s). Doron et al. also states, at col. 9, line 62, that RF coupling may be used when air gaps are present, such as in open surgery, as otherwise tissue impedes RF signal propagation.

Other procedures may also benefit from acquiring spatial knowledge during administration of a therapy to focus external energies to a desired targeted internal location, such as ultrasonic radiation treatments, microwave or RF ablation therapies, and localized ultrasonic or light activation of drugs.

Despite the foregoing, there remains a need for improved or alternate techniques for providing spatial data of target regions and/or implants.

### SUMMARY OF THE INVENTION

Certain embodiments of the present invention provide telemetric systems with implantable sensor units that can be used both as positional markers and to sense selected internal parameters.

In particular embodiments, the present invention provides telemetric systems with wireless sensors implanted at one or more target sites in the body, with the implanted sensors configured to generate signals that can be used to define sensor orientation and/or position in the body and detect radiation doses and/or internal temperature proximate the target site delivered to a target site during or after a radiation therapy session. The positional and radiation dose signal data can be serially and/or concurrently wirelessly relayed to an external reader or processor. For applications employing greater than one implanted sensor unit, each sensor unit can have a unique identifier and can be selectively serially polled or interrogated.

The sensor units can be configured to dynamically sense multiple tumor physiological and biological parameters, biophysical changes associated with tumors, and/or data allowing in situ substantially real-time measurements of radiation dose.

The sensor units can be used to provide dynamic spatial data or localization of a target region or implant that may be used to direct radiation therapies or gate delivery for radiation therapies such as EBRT (Electron Beam RadioTherapy). The sensor units may be configured to sense other or additional desired internal parameters, such as, but not limited to, temperature, localized RIT (radioimmunotherapy), drug uptake, the presence of localized radiolabeled or fluorescently labeled antibodies, and the like. Thus, the localization systems can be used in conjunction with other procedures such as laparoscopic, thorascopic, endoluminal, periviseral, and endoscopic procedures, cryosurgery, biopsies, mammographies, and ablation procedures using focused energy'such as RF, laser, and/or microwave.

In addition, unlike conventional implanted sensors, tumor monitoring systems used to monitor tumors can be exposed to a relatively harsh environment during a treatment protocol or strategy which can extend over a period of weeks, or even months (such as applied heat, chemicals and/or radiation). Further, such a harsh environment, coupled with an extended treatment period, can affect the function of the device and thus, potentially corrupt the measurement data it generates.

Certain embodiments are directed to target locating and in vivo sensor systems adapted for use with a therapy delivery source. The systems include: (a) an external solenoid member; (b) a mechanism operably associated with the external solenoid member, wherein, in operation, the mechanism is configured to move the solenoid external to the patient; (c) a controller configured to direct the movement of the mechanism, the controller being in communication with a power source configured to power the external solenoid; (d) at least one implantable sensor unit, wherein the at least one implantable sensor unit is configured to sense at least one predetermined parameter of interest in vivo, and wherein the at least one implantable sensor unit comprises a solenoid, and wherein, in operation, the sensor unit solenoid cooperates with the external solenoid to generate a coupling signal having a signal strength that varies based on the position of the external solenoid member relative to the implanted sensor unit; (e) a computer module in communication with the controller comprising computer program code that evaluates the coupling signal strength in relation to the position of the external solenoid and determines the position of the at least one sensor unit; and (f) an external reader configured to wirelessly communicate with the at least one implantable sensor unit to obtain data associated with the at least one predetermined parameter of interest.

In particular embodiments, the mechanism can be an articulate arm and the external reader can be configured to communicate with the implanted sensor unit using a bit encoded RF signal and the at least one sensor unit is configured to wirelessly relay an RF signal to the reader when implanted in a cancerous tissue treatment site. The external solenoid and the internal solenoid of the at least one sensor unit can be configured to cooperate to generate a detectable coupling signal at a depth of up to about 14 cm. The at least one sensor can be a plurality of discrete sensor units that may communicate with the external reader at the same frequency using unique bit encoded identifiers in the RF signal.

Other embodiments of the present invention are directed to methods of obtaining spatial data and radiation dose data regarding a target in vivo treatment site. The methods include: (a) implanting at least one sensor unit proximate and/or in a target treatment site of a patient; (b) sensing in vivo at least one predetermined parameter of interest using the implanted sensor unit; (c) wirelessly transmitting data associated with the sensed at least one parameter from the at least one sensor unit to an external reader; (d) providing an external coupling member located external of the patient proximate the target treatment site, the coupling member being configured to cooperate with the at least one implanted sensor to generate a coupling signal that varies in relation to the position of the coupling member with respect to the at least one sensor unit; (e) moving the coupling member; (f) detecting the signal strength of the coupling signal at a plurality of locations traveled based on the moving step; and (g) determining the position of the at least one sensor unit in the body based on the detecting step, thereby having the implanted sensor unit act as a positional marker and an in vivo sensor.

In particular embodiments, the method can also include: positioning the patient in an imaging system in a registered position; obtaining an image of the target treatment site and at least one implanted sensor with the patient in the registered position in an imaging system; aligning the coupling member to a fiducial marker on the imaging system relative to the registered position; and obtaining an electrical measurement of signal strength of the coupling signal while the patient is in the registered position and the coupling member is aligned to define the initial spatial position of the at least one sensor unit in three-dimensional space.

Other embodiments are directed toward computer program products for obtaining spatial data regarding the position of at least one implanted sensor. The program includes: computer readable storage medium having computer readable program code embodied in the medium, the computer-readable program code comprising computer readable program code for determining the spatial location of a selected one of the at least one implanted sensor units using input data associated with variation in signal strength of a coupling signal generated by an external solenoid and the at least one sensor unit over different known external positions of the external solenoid.

The computer product may also be configured to analyze radiation dose data using the at least one implanted sensor, and the program can include computer readable program code for determining the radiation dose detected by the implanted sensor unit and computer readable program code for commencing wireless data transmission from the at least one implanted sensor unit. As noted above, the at least one sensor unit can be positioned in a subject undergoing treatment for cancer.

Advantageously, the systems, methods, and devices of the present invention can act as positional markers to generate spatial localization data and may monitor, in real time and/or dynamically, radiation dose, temperature, and/or specific indices associated with tumor physiology or other desired internal parameters. For the radiation therapy, the sensed data can be made to be available during the radiation therapy session for use in treatment decisions. Embodiments of the present invention may be particularly suitable for oncology applications.

### BRIEF DESCRIPTION OF THE DRAWINGS

FIG. 1 is a block diagram of operations that may be used to carry out spatial positioning and radiation dose evaluations according to embodiments of the invention.

FIG. 2 is a block diagram of operations that may be used to carry out spatial localization evaluations of a target site according to embodiments of the present invention.

FIG. 3 is a block diagram of operations that may be used-to carry out spatial positioning evaluations and obtain data of selected internally sensed parameters according to embodiments of the present invention.

FIG. 4A is a schematic illustration of a subject in position for a radiation therapy session and being evaluated for internal spatial data according to embodiments of the present invention.

FIG. 4B is a front view of an implantable sensor unit according to embodiments of the present invention.

FIG. 5 is a schematic illustration of a system with an implantable sensor unit that can be used as both a positional marker and to actively sense one or more internal selected parameters of interest according to embodiments of the present invention.

FIG. 6 is a block diagram of a system for obtaining spatial and sensed data according to embodiments of the present invention.

FIG. 7 is a block diagram of a data processing system and/or computer modules according to embodiments of the present invention.

FIG. 8 is a front view of an external solenoid and a smaller internal (implantable) solenoid.

FIG. 9 is a graph of an example of applied voltage (larger amplitude wave) and response voltage (smaller amplitude wave) with frequency adjusted to provide suitable coupling.

FIG. 10 is a graph of coupling voltage (mV) as a function of separation distance (cm) according to embodiments of the present invention.

FIG. 11 is a front view of two separated solenoids, with the small solenoid offset at an angle with respect to the orientation illustrated in FIG. 8.

FIG. 12 is a front view of the two solenoids shown in FIG. 11, with the small solenoid offset at-yet a different angle relative to that shown in FIG. 11 or FIG. 8.

### DETAILED DESCRIPTION OF EMBODIMENTS OF THE INVENTION

The present invention now will be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. In the drawings, like numbers refer to like elements throughout, and thickness, size and dimensions of some components, lines, or features may be exaggerated for clarity. The order of operations and/or steps illustrated in the figures or recited in the claims are not intended to be limited to the order presented unless stated otherwise. Broken lines in the figures, where used, indicate that the feature, operation or step so indicated is optional.

Generally stated, the systems, devices, methods and computer program products of the present invention are aimed at determining where implantable sensor units are spatially located (localization) in the body to act as a fiducial marker at various points in time using a desired imaging and/or spatial locating modality such as, but not limited to, MRI, CT, X-ray, RF, ultrasound, or other locating means. In certain embodiments, the sensor units are both positional markers and configured so that they are capable of detecting predetermined parameters in vivo. The sensor units can be configured to detect changes of selected parameters associated with internal activity, pathology, physiology, and/or kinetics of living systems.

In particular embodiments, the sensor units may be configured to detect or sense radiation doses delivered to and/or the temperature at or proximate to one or more target sites. The radiation dose may be delivered by an external beam therapy system. The target site may be any internal region undergoing analysis or therapy, and may be a site associated with tumors such as cancerous tissue. That is, the site may be a localized cancerous tumor or a site where the tumor has been excised, but the excision site and/or tissue proximate thereto is the target tumor site. In certain, embodiments, non-target sites may also be monitored for selected internal parameters or conditions. In particular embodiments, sensitive or non-target sites may be monitored to detect radiation dose received thereat, as desired. The radiation dose may be detected using additional implanted sensor units or disposable radiation sensor patches. For additional discussion of suitable disposable (typically single-use) radiation sensor patches, see co-pending U.S. patent application Ser. No. 10/303,591, filed Nov. 25, 2002, the contents of which are hereby incorporated by reference as if recited in full herein.

More specifically, certain embodiments of the present invention are directed to operations that determine the in situ location of implanted sensors that are adapted to detect at sufficient intervals, one or more of: (a) radiation received internally to a target tumor site and/or to non-targeted healthy tissue or sensitive sites; (b) oxygen; (c) pH; (d) cell proliferation of any organ or tumor system; and/or (e) the molecular and cellular determinants of sensitivity or resistance to cytotoxic or therapeutic agents. The monitoring system and associated sensor units can provide the data for the sensed parameter under “normal” physiological conditions, in situ, as well as prior to, during and following any perturbation (such as, but not limited to, radiation, chemical or cytotoxic stimuli, thermal treatments, and hyperthermia and/or hypothermia) of such systems.

As such, the monitoring systems and methods of the present invention can be useful in many applications, such as, for example, pulmonary, gastrointestinal, neuroscience and pre-clinical research. Nonetheless, the present invention is believed to have particular importance and suitability for in vivo tumor systems.

In certain embodiments, the sensor units can be implanted relatively deep in the body of the subject and may remain in the body for a 2-6 week period or even longer in order to provide in vivo evaluation and monitoring of tumors prior to, during, and subsequent to an active treatment, and preferably over an entire treatment regime or period. As such, the internal in situ sensors of the present invention are preferably configured to be biocompatible and provide a service life suitable for episodic treatment evaluation of at least about 4-6 weeks, and more preferably at least about 6-10 weeks, and still more preferably at least about 10-12 weeks, whether exposed to radiation, chemotherapy, heat or ionic electric fields (such as the treatment provided by a Thernotrong system) directed to the tumor. Additional description of suitable sensor units is found in U.S. Pat. No. 6,402,689, and co-pending, co-assigned U.S. patent application Ser. No. 10/127,207, and will be discussed further below. The sensor units are configured with internally mounted electronics that wirelessly communicate with an external reader. The sensor units can be configured as a miniaturized elongate (medical grade glass encapsulated or suitable aluminosilicate material) sensor body having a length of about 25 mm or less and a width of about 3 mm or less. The sensor body may be radio-opaque or use radio-opaque markers or coatings for visibility on CT or X-ray scans.

The sensor units can be configured as radiation sensors that can be used to verify irradiation doses delivered during photon irradiation treatment (cumulatively, typically in the range of between about 3000-6000 cG, with each treatment optionally being increments of the total, such as 200-500 cG). Thus, use of a radiation monitor during real time delivery can help control a more precise delivery dose of gamma radiation to the tumor site. Data regarding the distribution of dose within the tumor following photon irradiation and/or verification of a calculated or planned dose, may be particularly of interest as complex beam shaping, high dose conformal therapy, or beams at oblique angles may not consistently deliver the planned dose. In certain embodiments, the sensor units may be B radiation monitors used to monitor radioactively labeled compounds, drug uptake and/or utilization, blood flow in the tumor, sensitivity to specific drugs, drug distribution, labeled-glucose (or bio-constituent or metabolite thereof) or another analyte of interest in various locations or organs (as well as cell proliferation as discussed above).

Patients according to the present invention can be any animal subject, and are preferably mammalian subjects (e.g., humans, canines, felines, bovines, caprines, ovines, equines, rodents, porcines, and/or lagomorphs), and more preferably are human subjects.

The present invention is described herein with reference to flowchart illustrations and/or block diagrams of operations, methods and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, embedded processor or other programmable data processing apparatus to produce a machine, such that the instructions, which execute via the processor of the computer or other programmable data processing apparatus, create means for implementing the functions specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart and/or block diagram block or blocks.

As will be appreciated by one of skill in the art, the present invention may be embodied as a system, method, data or signal processing system, or computer program product. Accordingly, the present invention may take the form of an entirely software embodiment or an embodiment combining software and hardware aspects. Furthermore, the present invention may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium, upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of the present invention may be written in an object oriented programming language such as Java7, Smalltalk or C++. However, the computer program code for carrying out operations of the present invention may also be written in conventional procedural programming languages, such as the “C” programming language or even assembly language. The program code may execute entirely on the user's computer, partly on the user's. computer, as a stand-alone software package, partly on the user=s computer and partly on a remote computer or entirely on the remote computer. In the latter scenario, the remote computer may be connected to the user=s computer through a local area network (LAN) or a wide area network (WAN), or the connection may be made to an external computer (for example, through the Internet using an Internet Service Provider).

The flowcharts and block diagrams used herein illustrate systems and/or operations that can be used to obtain and/or analyze localization signal data of telemetric (wirelessly operated) implant sensor units. The analysis of the localization signal data can be carried out to provide real-time data on spatial location (i.e., movement) of a tumor during active delivery of a therapy such as, but not limited to, external beam radiation therapy. This spatial data can be interfaced with a delivery system to control the delivery of the therapy. For example, the spatial data can be used with a radiation (external beam) therapy system to thereby control, direct, guide, and/or gate (gate meaning direct the “on” or “off ” of a radiation beam transmitted into the body) during an external beam radiation therapy session. The flowcharts and block diagrams used herein also illustrate systems and/or operations that telemetrically obtain and/or analyze data associated with radiation measurements from the telemetric implant sensor units. In this regard, each block in the flow charts or block diagrams represents a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may be executed substantially concurrently or the blocks may sometimes be executed in the reverse order, depending upon the functionality involved.

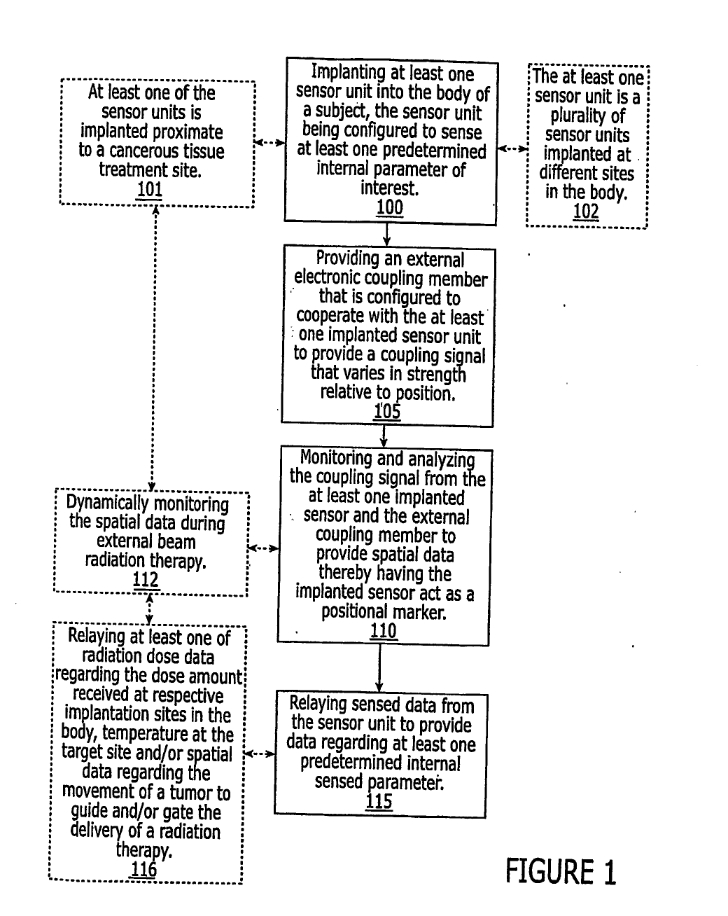

Turning now to the figures, FIG. 1 illustrates operations that can be performed to carry out embodiments of the present invention. At least one sensor unit can be implanted into the body of a subject, the sensor unit can be configured to sense at least one predetermined internal parameter of interest (block 100). At least one of the sensor units can be positioned proximate a cancerous tissue treatment site (block 101). The at least one sensor unit can be a plurality of discrete sensor units implanted at different sites in the body of the subject (block 102). The different sites can all be proximate the cancer treatment site (dispersed thereabout or at different depths into the target treatment site) or one or more may be placed proximate normal tissue. An external wireless electronic coupling member can be provided so that the coupling member cooperates with the at least one sensor unit to provide a coupling signal that varies in strength relative to the position of the external coupling member to the implanted sensor unit (block 105). The coupling signal is monitored and analyzed to provide spatial data regarding the position of the sensor unit (and hence, in certain embodiments, the cancerous tissue site or tumor) so that the sensor unit acts as a positional marker (block 110). The spatial data can be dynamically monitored during external beam radiation therapy to allow the therapy to be guided and/or gated based on the dynamic spatial data (block 112). Sensed data associated with at least one predetermined internal parameter can be wirelessly relayed from the implanted at least one sensor unit (block 115). The sensed data can be radiation dose and/or temperature data as sensed from respective implantation sites in the body of the subject and transmitted externally therefrom (block 116).

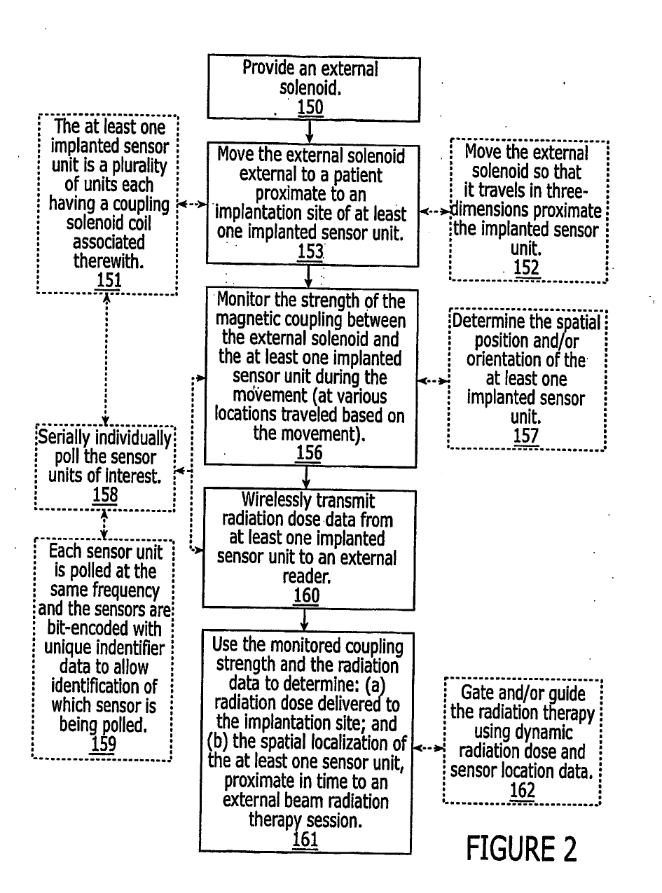

FIG. 2 illustrates operations that can be performed to carry out embodiments of the present invention. As shown, an external solenoid can be provided (block 150).

The external solenoid can be moved external to the patient and proximate to an implantation site of at least one sensor unit (block 153). The at least one sensor unit can be a plurality of sensor units, each having a coupling solenoid associated therewith (block 151). The external solenoid can be moved so that it controllably travels in three dimensions proximate the implanted sensor unit (block 152). The signal strength of the magnetic coupling between the external solenoid and the sensor unit solenoid can be monitored during the translation (block 156). The spatial position and/or orientation of the implanted sensor unit can be determined (block 157) based on the translating and monitoring steps.

In particular embodiments, radiation dose data can be wirelessly transmitted from the at least one sensor unit to an external reader (block 160). The monitored coupling strength and the radiation dose data can be used to determine: (a) the radiation dose delivered in situ to the target site; and/or (b) the spatial localization of the at least one sensor unit proximate in time to an external beam radiation therapy session (block 161). The radiation and/or spatial data can be collected dynamically and used to deliver, guide, and/or gate the radiation therapy (block 162). The spatial data can also be used to guide other therapies or surgeries.

Where multiple sensor units are employed, each sensor unit of interest can be selectively serially (individually) polled (block 158). Each sensor unit can be polled at the same (RF) frequency using bit-encoded unique identifiers in the operation of each sensor unit (block 159).

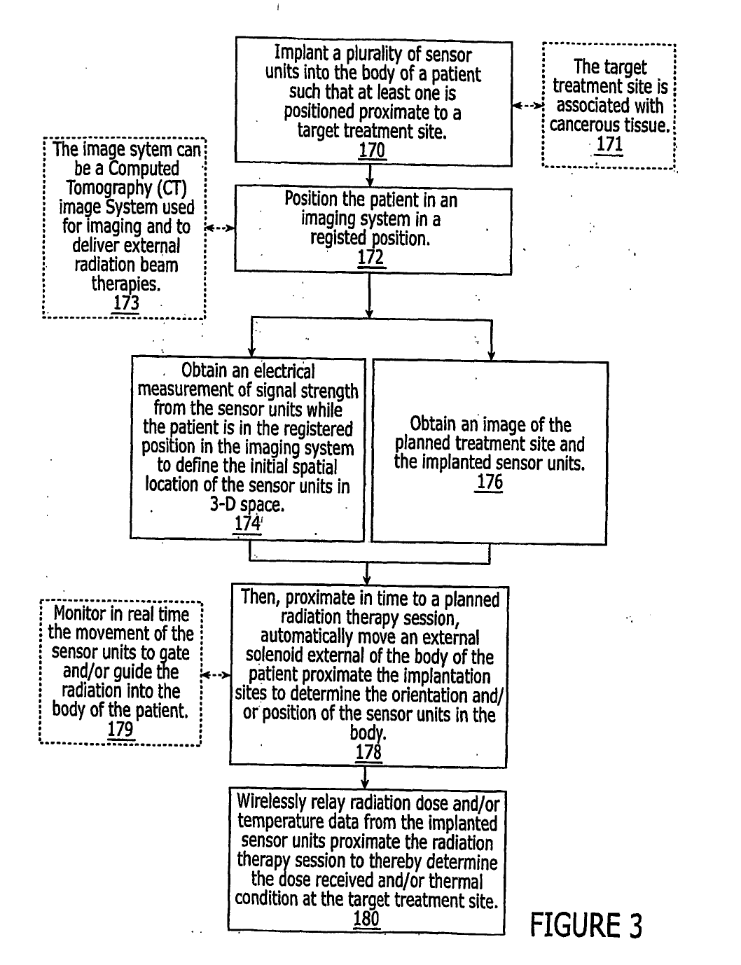

FIG. 3 illustrates additional operations that can be performed to carry out embodiments of the present invention. A plurality of probes such as radiation (dosimeter) sensors can be implanted in the body of a patient such that at least one is positioned proximate a target treatment site (block 170). The target treatment site can include a site associated with treatment of cancerous tissue (block 171). The patient can be positioned in an imaging system in a registered position (block 172). The imaging system may be a Computed Tomography (CT) image system used for imaging and to deliver external radiation beam therapies (block 173). Other radiation/imaging systems may also be used. An electrical measurement of signal strength can be obtained from the sensor units while the patient is in the registered position in the imaging system to define an initial spatial location of the sensor unit(s) in three-dimensional (free) space (block 174). An image of the planned treatment site with the implanted sensor units held thereat can be obtained (block 176). The image can be obtained while the patient is in the registered position. Each of the initial evaluations (image and electric measurement) can be obtained during a radiation planning session. Then, proximate in time to a planned radiation therapy session, an external solenoid can be automatically moved and/or translated external of the body of the patient but proximate to the implantation site(s) to obtain spatial data and determine the position and/or orientation of the sensor units in the body (block 178). The movement of the sensor units (and hence, target site, can be monitored in real time (dynamically) using the spatial data to, in turn, guide and/or gate (meaning switching the radiation beam “on” and/or “off”) to control the radiation delivered into the body of the patient (block 179).

The sensed data (ie., radiation and/or temperature and the like) can be wirelessly relayed from the implanted sensor units to an external location or locations during a radiation therapy session to thereby determine the dose received at the target treatment site(s) or other location (block 180).

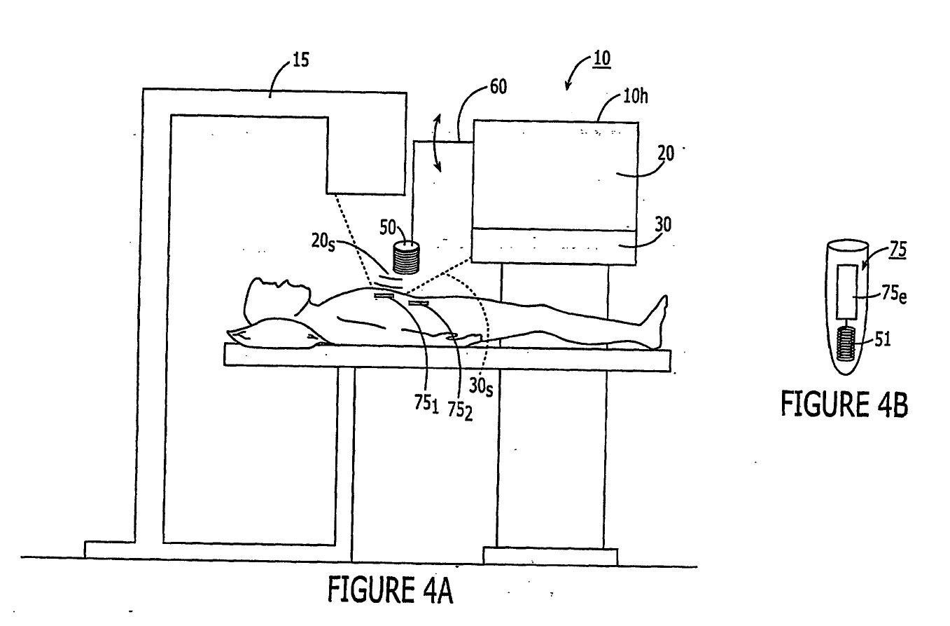

FIG. 4A illustrates a system 10 for obtaining intra-body spatial data that may be used as a radiation therapy guide system. As shown, the system 10 includes an external solenoid 50, a mechanism 60 for controllably moving the external solenoid. The mechanism 60 can be an articulating arm 60 that holds and controllably translates the solenoid 50 (as schematically shown by the arrow in the figure). Other mechanisms can also be used as will be known to those of skill in the art. The mechanism 60 can be numerically controlled and/or operated by robotic-control or assist systems. As also shown, the system 10 includes a spatial data controller or processor 20, one or more implanted telemetric sensor units (shown as two units 75₁, 75₂), and an external reader 30 configured to receive wireless transmissions corresponding to data for at least one sensed internal parameter from the implanted sensor units 75. As shown, the system 10 can include a primary housing 10h. The arm 60 may be attached to the housing 10h or the arm 60 may be held by another support member (not shown) and the powering of the solenoid 50 and the translation and movement pattern thereof can be controlled (numerically controlled (N/C) or robotically controlled) based on directions from the controller 20. The housing 10h may include user input controls and a display or other communication means as desired (not shown).

In operation, the sensor units 75 are configured with electronics 75e (FIG. 4B) that allow them to communicate with the external reader 30 using RF signals 30s to relay data associated with at least one internally sensed parameter of interest. The sensor units 75 also are configured to electrically cooperate with the external solenoid 50 to generate a wireless coupling signal 20s when the external solenoid 50 is sufficiently proximate one or more of the implanted sensor units 75. The sensor units 75 may be inductively powered. The sensor units 75 can be configured with a solenoid 75s (FIG. 4B) that is substantially smaller than the external solenoid 50. The sensor solenoid may also be used to inductively power the implanted sensor as well as to provide the spatial coupling signal. Each respective sensor unit 75 may be operated so as to generate the two signals 20s, 30s serially or concurrently.

The external reader 30 and the spatial data processor 20 may be housed in the same primary housing unit (as shown) and may share the same or portions of the same data or computer processing system. In other embodiments, the external reader 30 and the spatial data processor 20 are separate units that operate independently and can be held in different housings. For additional description of the sensor unit 75 and reader 30, see U.S. Pat. No. 6,402,689 and co-pending U.S. patent application Ser. No. 10/127,207; the contents of these documents are hereby incorporated by reference as if recited in fill herein. Suitable sensor units and telemetric readers are under evaluation by the U.S. Food and Drug Administration (“FDA”) and it is contemplated that they will be available from Sicel Technologies, Inc., located in Morrisville, N.C., in the future.

The external solenoid 50 is a coil of wire wrapped over a substrate material. The external solenoid 50 may have a hollow core or a solid core, or a core with hollow spaces formed therealong. The conductive coil has a wire size and number of wraps that are selected to generate a magnetic signal that produces a coupling signal from selected implanted sensor units 75 at a clinically useful coupling depth in the body. The coupling depth may be between about 5 cm-11 cm, and is typically about 5-12 cm. In certain embodiments, the solenoid 50 may be formed of a ferrite coil.

In operation, AC or DC current is applied to the external solenoid 50. In certain embodiments, the current is AC (such as a sine wave) with a frequency that is between about 100 kHz and 1 MHz, and in certain embodiments, between about 500 kHz and 1 MHz. The articulated arm 60 translates the external solenoid 50 over the patient and proximate to a selected implant site(s). A response coupling signal 20s is generated by the implanted sensor unit 75 as the external solenoid 50 approaches and moves away. The coupling signal 20s has a signal strength associated with the strength of the magnetic coupling that varies (typically in the mV range) with relative position of the targeted implanted sensor unit 75. The arm 60 translates the external solenoid 50 in a selected pattern. The signal strength of the coupling signal 20s versus position of the solenoid 50 defined relative to a predetermined coordinate system is monitored. The positional data and associated coupling signal strength can be analyzed to define a unique trajectory that establishes the maximal signal strength. The maximal value of the signal itself may not be important, as the relative maximal value is used to define the spatial location of the sensor unit. Nonetheless, if the maximal value is significantly different from an a priori or predicted value, an error or alert as to the presence of a potential abnormality may be noted to allow a clinician to take remedial actions (such as to reinitiate the operations). The unique trajectory can be used to define at least one of spatial position and/or orientation. In certain embodiments, the arm 60 translates the solenoid through a controlled three-dimensional travel path while the coupling signal strength 20s is monitored.

In certain embodiments, an initial spatial evaluation can be performed during the planning session and/or before each radiation therapy session and the coupling, signal strength of one or more implanted sensor units 75 can be monitored during the radiation therapy to provide dynamic spatial data regarding any movement of the sensor unit. Translation of the solenoid 50 may not be required as most movements (or coupling depth variation) will be relatively small and the coupling signal strength can be used to define the spatial data. Further, coupling strength orientation and separation distance (and hence location) can be determined mathematically using a suitable mathematical model, such as Maxwell's equations and/or a third-order polynomial equation, and/or with a few signal strength measurements at selected locations without requiring a full three-dimensional trajectory of empirical measurements.

The coupling signal strength may be compared to the signal strength positional trajectory established at the start of the radiation therapy session to define the actual spatial location.

Alternatively, the radiation therapy can be gated in substantially real time to “off” and/or “on” upon detection of movement or change in coupling signal associated with a change in coupling separation (change in signal strength) of the sensor unit. In this embodiment, the sensor unit 75 and the solenoid 50 may not move, but the coupling separation may vary, as during respiration or urinary discharge when the tissue or organ that the sensor unit is implanted in may move. Similarly, delivery of the radiation therapy can be guided based on spatial data provided by the implanted sensor units to focus the radiation beam to projections defined by dynamic boundaries (depth, width, volume) established by the evaluation. In other embodiments, a shortened translation pattern (1-D, 2-D, or 3-D) can be used to evaluate position of the sensor unit(s) 75, and thus, the target site, during the therapy itself

In operation, the response signal is analyzed based on its response at various locations to predicted responses based on orientation and separation distance. The response signal can be compared to predicted norms for symmetry, asymmetry, maxima location(s), null (if any), and the like. For example, the signal response curve can be analyzed to determine when the external solenoid is perpendicular to the sensor unit coil and whether the sensor coil is directly in-line with the external solenoid (where the largest signal strength can occur in this orientation). In addition, in operation, in two different relative orientations, the coupling signal can have two maxima, each located on opposing sides of a null (the null associated with when the external coil is directly in line with the implanted sensor unit coil). However, in each of the different orientations, the symmetry of the peak amplitude response may differ, and the unique response to various angular orientations of the external solenoid can be used to determine the orientation and/or location of the implanted sensor unit.

FIG. 4B illustrates one example of an implantable sensor unit 75. The sensor unit 75 includes electronics 75e configured to allow wireless communication to the external reader 30. The sensor unit 75 can include a RADFET that operates with a threshold voltage “Vth” shift that is proportional to absorbed radiation dose. The sensor unit 75 can be inductively powered via an inductively coupled internal coil. The sensor unit 75 can be held in a hermetically sealed glass capsule or other medically suitable material that is substantially impermeable. The sensor unit electronics 75e can include a micro- (or nano-) processor controller that controls data acquisition and reader/sensor unit communications that can be mounted on a ceramic substrate. The electronics 75e can include custom chip designs with routings to semiconductor chips provided for data acquisition. The sensor unit 75 can include a bidirectional antenna. The sensor unit 75 can be configured with digital communication components using a 12-bit data acquisition that can provide about a 1 mV resolution (or better) of the Vth measurement and may operate with a 16-bit CRC error checking capacity. The electronics 75e can be potted in Class VI USP epoxy and hermetically sealed inside a glass capsule. The external surface or body of the capsule can be coated with a Parylene C material or other biocompatible coating. The sensor unit 75 can be EO sterilized and adapted to be suitable for chronic in vivo implantation as described. As noted above, the sensor body or portions thereof, may be radio-opaque for visual contrast in CT scans and port films and the like. Additional description of exemplary sensor unit housing configurations can be found in co-pending U.S. patent application Ser. No. 10/353,857, the contents of which are hereby incorporated by reference as if recited in full herein.

In certain embodiments, the system 10 can be configured to individually selectively (serially) poll, address, and/or interrogate a selected implanted sensor unit 75. The sensor units 75 can be configured to operate or communicate with the reader at the same frequency. To control and/or identify which sensor unit 75 is in active communication mode, a single or multi-bit identifier can be generated and used in the data stream.

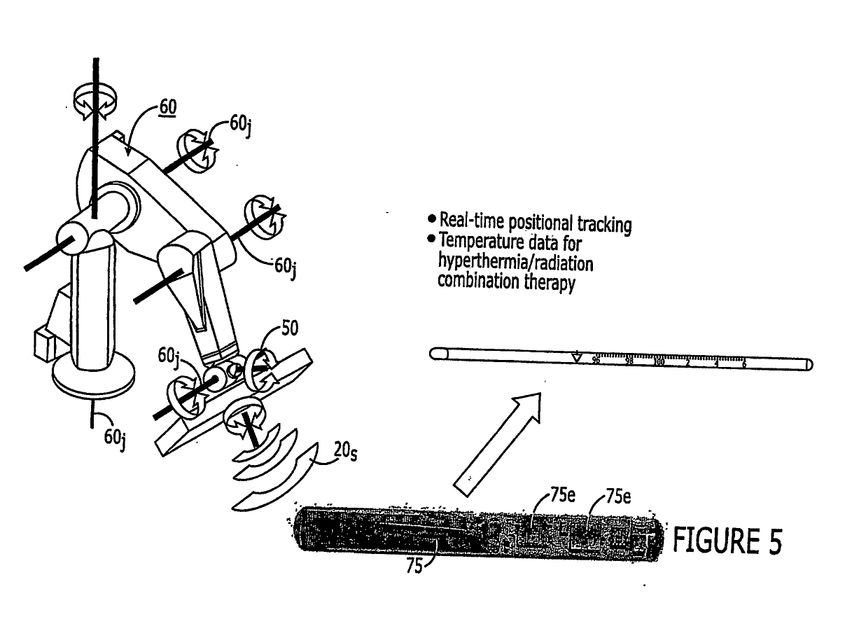

FIG. 5 illustrates that, in certain embodiments, at least one of the implanted sensor units 75 can be configured to monitor both radiation dose and temperature. The temperature sensor shown is a mercury based thermometer for illustration purposes only. In operation, a digital temperature sensor may be included in the sensor unit 75 itself. The system 10 can be configured to provide real-time positional tracking of one or more of the sensor units 75, and hence, the target (tumor) site. The temperature data and radiation dose data can be used to help administer hyperthermia/radiation combination therapies. The sensor itself can be located at any desired depth in the body.

FIG. 5 illustrates one example of an articulated arm 60 that can be used to automatically translate the solenoid 50. As used herein, the term “articulated arm” means an arm with at least one pivot and/or rotational joint. As shown in FIG. 5, the articulated arm 60 has a plurality of joints 60j that are configured to allow for multi-directional, and typically, three-axis, three-dimensional controlled movement of the solenoid 50. The solenoid 50 may be translated through a predetermined 3-D trajectory during the spatial evaluation of one or more of the implanted sensor units 75. Articulated arms and robotic control systems are well known to those of skill in the art. Exemplary articulated arms and robotic control systems are described in U.S. Pat. Nos. 6,330,467; 6,309,397; 6,195,577; 5,770,834; 5,519,814; 5,303,384; 5,268,837; 5,263,809; and 5,243,477. The contents of these patents are hereby incorporated by reference as if recited in full herein.

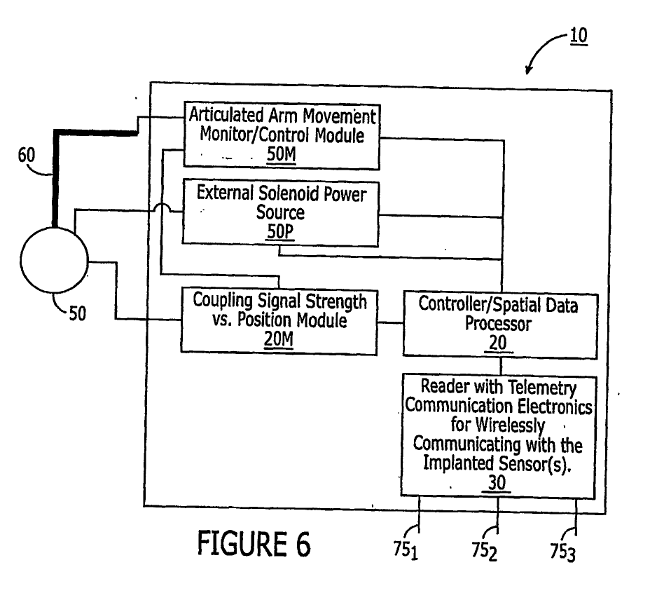

FIG. 6 is a schematic illustration of operational components of a system 10 having the capacity to communicate with one or a plurality of telemetric bi-functional implanted sensors 75. The sensors 75 are used as fiducial markers and to actively sense selected internal parameters as discussed above. As shown, the system 10 includes a power source for the external solenoid 50P, and a (spatial data) controller or processor 20 in communication with a coupling signal strength versus position monitor module 20M and also with an articulated arm movement controller module 50M. The coupling strength monitoring module 20M and the articulated arm movement module are in communication with the arm 60 and/or the solenoid 50. The solenoid 50 can include a pick-up coil or voltage probe sensor used to detect the signal strength (volts) of the magnetic coupling signal.

The coupling signal strength probe may be mounted proximate to the external solenoid 50 and can be in communication with the controller 20 and/or signal strength monitoring module (20M, FIG. 6). As noted above, the reader 30 may be in communication with the spatial data system controller 20, or may be configured so as to operate independently thereof so as to have the capacity to serially wirelessly communicate with a plurality of implanted sensor units 75 (shown as sensor units 75₁, 75₂,75₃).

As shown in FIG. 4A, in certain embodiments, the patient may be positioned in a registered position in an imaging system 15. The imaging system 15 may be configured to deliver a radiation therapy and may be configured as any suitable system therefore, as is well known to those of skill in the art. In certain particular embodiments, the imaging system 15 may be a computed tomography (CT) system. Other suitable radiation treatment systems include, but are not limited to, three-dimensional conformal external beam radiation, intensity modulated radiation therapy (IMRT), a “gamma knife” that employs a highly focused gamma ray radiation obtained from crossing or collimating several radiation beams, stereotactic radiosurgery and brachytherapy systems.

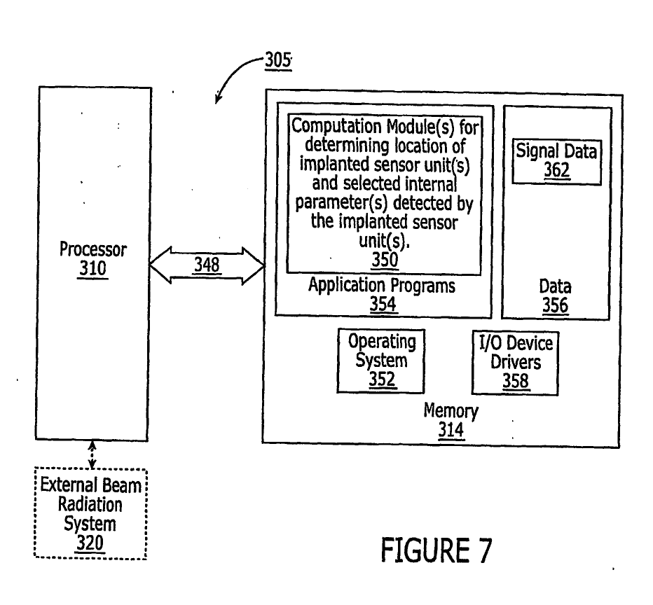

FIG. 7 is a block diagram of exemplary embodiments of data processing systems that include a computation module 350 in accordance with embodiments of the present invention. The processor 310-communicates with the memory 314 via an address/data bus 348. The processor 310 can be any commercially available or custom microprocessor. The memory 314 is representative of the overall hierarchy of memory devices containing the software and data used to implement the finctionality of the data processing system 305. The memory 314 can include, but is not limited to, the following types of devices: cache, ROM, PROM, EPROM, EEPROM, flash memory, SRAM, and DRAM.

As shown in FIG. 7, the memory 314 may include several categories of software and data used in the data processing system 305: the operating system 352; the application programs 354; the input/output (1/0) device drivers 358; a computation module 350; and the data 356. The computation module 350 includes computer program code that evaluates spatial location of at least one implanted sensor unit and at least one internal selected parameter detected by the at least one implanted sensor. In certain embodiments, the selected internal parameter is radiation dose detected by the at least one implanted sensor unit. In certain embodiments; the computation module 350 can be used during delivery of focused therapies, such as radiation therapies and can include real-time spatial data feedback that can identify whether the target tumor is moving.

The data 356 may include signal data 362 which may be obtained directly from the implanted sensor units and/or data acquired through a location determination module that analyzes coupling strength versus position. The processor 310 may communicate with an external beam radiation therapy delivery system 320.

As will be appreciated by those of skill in the art, the operating system 352 may be any operating system suitable for use with a data processing system, such as OS/2, AIX or OS/390 from International Business Machines Corporation, Armonk, N.Y., WindowsCE, WindowsNT, Windows95, Windows98, Windows2000, WindowsXP or Windows XT from Microsoft Corporation, Redmond, Wa., PalmOS from Palm, Inc., MacOS from Apple Computer, UNIX, FreeBSD, or Linux, proprietary operating systems or dedicated operating systems, for example, for embedded data processing systems.

The I/O device drivers 358 typically include software routines accessed through the operating system 352 by the application programs 354 to communicate with devices such as I/O data port(s), data storage 356 and certain memory 314 components and/or the image acquisition system 320. The application programs 354 are illustrative of the programs that implement the various features of the data processing system 305 and preferably include at least one application that supports operations according to embodiments of the present invention. Finally, the data 356 represents the static and dynamic data used by the application programs 354, the operating system 352, the I/O device drivers 358, and other software programs that may reside in the memory 314.

While the present invention is illustrated, for example, with reference to the computation module 350 being an application program in FIG. 7, as will be appreciated by those of skill in the art, other configurations may also be utilized while still benefiting from the teachings of the present invention. For example, the module 350 may also be incorporated into the operating system 352, the I/O device drivers 358 or other such logical division of the data processing system 305. Thus, the present invention should not be construed as limited to the configuration of FIG. 7, which is intended to encompass any configuration capable of carrying out the operations described herein.

The I/O data port can be used to transfer information between the data processing system 305 and the system 320 or another computer system or a network (e.g., the Internet) or to other devices controlled by the processor. These components may be conventional components such as those used in many conventional data processing systems, which may be configured in accordance with the present invention to operate as described herein.

While the present invention is illustrated, for example, with reference to particular divisions of programs, functions and memories, the present invention should not be construed as limited to such logical divisions. Thus, the present invention should not be construed as limited to the configuration of FIG. 7 but is intended to encompass any configuration capable of carrying out the operations described herein.

Particular embodiments of the invention will now be described in the following non-limiting examples.

### EXAMPLES

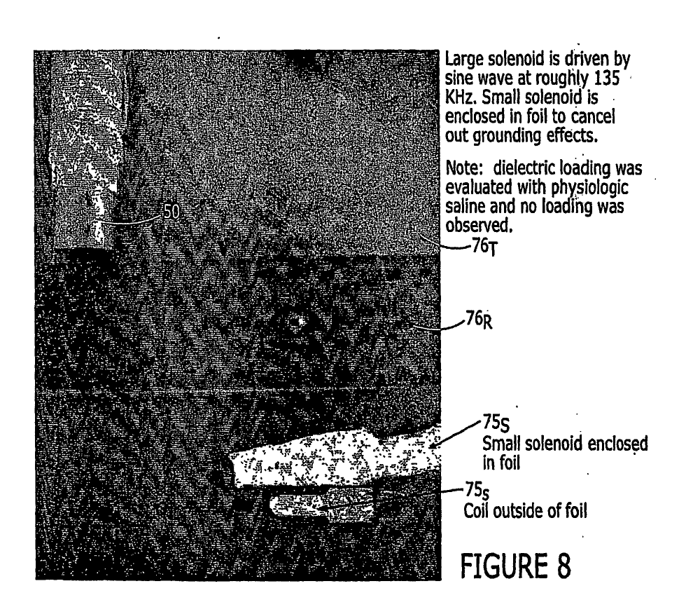

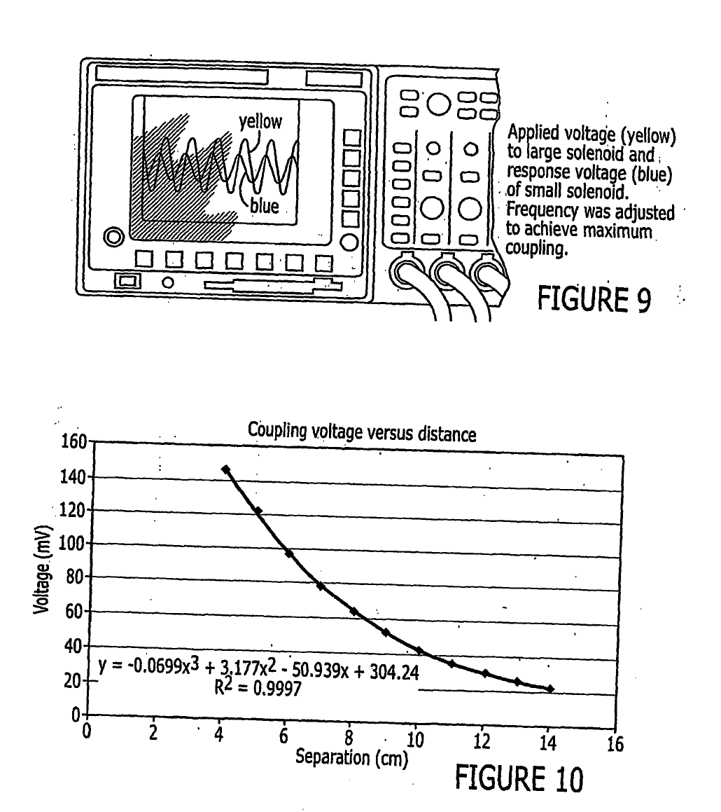

Empirical measurements were taken with an implantable sensor unit having an 10 internal coil positioned at different orientations and coupling distances away from an external solenoid coil. The experimental set-up is shown in FIG. 8 and a typical probe-response set of waveforms (coupling signals) is shown in FIG. 9. As shown in FIG. 8, a ruler 76R was laid on a table-top surface 76T for positional reference. A first measurement was taken with the external and sensor coils parallel to each other with the sensor unit coil being positioned approximately centrally of the external larger coil. In this orientation, the coupling is maximal when the sensor coil is substantially centered in this manner. FIG. 10 illustrates the relationship between separation distance (cm) and induced voltage (mV). FIG. 9 illustrates an applied signal (from the external solenoid) and a response (coupling) signal. The applied voltage is the larger amplitude wave signal and the response or coupling signal is the smaller amplitude wave signal. In this evaluation, the frequency was adjusted to increase coupling signal strength. FIG. 10 also illustrates an equation used to curve-fit the function for separation distance versus signal strength: y=−0.0699x³+3.177x²−50.939x+304.24, where R²+0.9997.

FIG. 8 illustrates the solenoids positioned at 90 degrees. The external solenoid 50 was moved along a ruler laid flat on a table and the voltage it induced in a pickup coil was noted as a function of its position relative to the sensor solenoid 75s.

The external solenoid 50 was driven by a sine wave excitation or input signal at about 135 kHz. The small sensor solenoid 75s was enclosed in foil to cancel out grounding effects in one evaluation and exposed external of the foil for another as shown in the two adjacent sensor units shown in FIG. 8. The dielectric loading was evaluated with physiologic saline and no loading of relevance was observed. FIG. 8 illustrates the position of greatest coupling strength where the two coils are perpendicular to each other. As the external coil is moved, the response voltage traces out one maximum (at about the position shown), a minimum or “null,” and a second maximum at a distance that is the same as the first maximum from the null location.

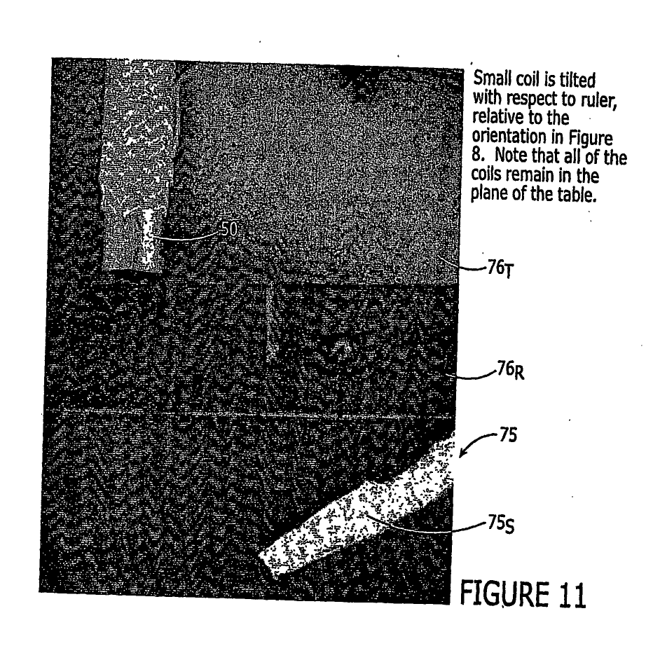

FIG. 11 illustrates the sensor coil 75s and sensor 75 tilted with respect to the ruler relative to the orientation shown in FIG. 8. In this evaluation, each coil remains in the plane of the table on which the ruler and coils lie. The sensor coil 75s is tilted at an angle with respect to the direction of travel of the external coil. When the external coil is moved along the ruler, there are again two maxima and a null. The left maximum had a peak that was smaller than the right maximum and the distance from the left maximum to the null position was less than the distance from the right maximum to null position. Thus, the symmetry (or asymmetry) of the peaks (amplitude) and distance to the null position allow for relative orientation of the two coils in the plane of the table to be unambiguously identified.

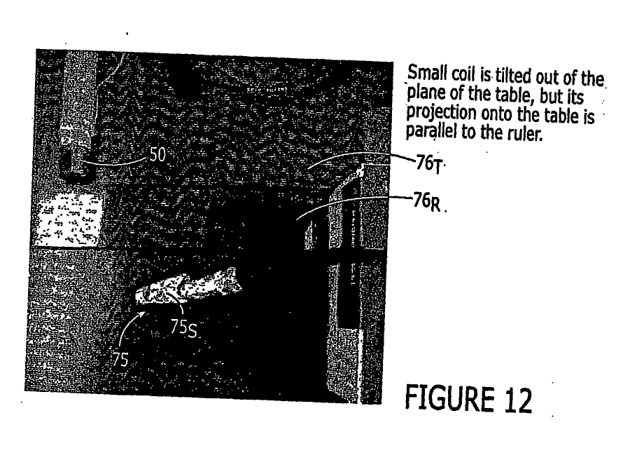

FIG. 12 illustrates the sensor coil 75s being tipped out of the plane of the table, but with its projection onto the table parallel to the ruler. In this configuration, as the external coil is moved along the ruler (still in the plane of the table and perpendicular to the ruler as was the case in the evaluation configurations shown in FIGS. 8 and 11), there are two maximum locations and a null location. As before the null occurs when the external solenoid is in line with the sensor coil. The left maximum is smaller that the right maximum, and in this evaluation, the right and left maximum were the same distance from the null position. The maximum on the left was more flat than the right.

In addition, with the sensor coil oriented to tilt up further out of the plane of the table 90 degrees, the movement of the external coil resulted in just one maximum when in line with the sensor coil (previously a null location) and fell off symmetrically on either side thereof.

As described above, an image the position of the implanted sensor unit(s) can be obtained using CT or other imaging modality during a planning session. The positional tracking of the articulated arm can be carried out to allow positional or spatial data to be mapped relative to the image. Once the initial position of the sensor unit is established, the external solenoid can be translated along a substantially straight track or travel path, such as described for the evaluation carried out in FIG. 8. The value of the maxima can be recorded (manually or automatically) to establish a reference distance metric. As noted above, the value of the maxima can be used for future evaluations of sensor unit position in the body, as it can be the reference coupling value for future spatial location evaluations.

At a therapy session, the external solenoid can be scanned (signal strength versus position) along the same trajectory as the set-up trajectory. If the implanted sensor unit remains in its original position and orientation, the same trajectory will give the same trace signal symmetry and same maxima value(s). If the implanted sensor unit has twisted in its plane then the signal response of FIG. 11 will occur. If the implanted sensor unit has titled up out of the original plane, the situation discussed with respect to FIG. 12 will occur. This spatial data will likely be of interest to assure that the therapy beam is not then directed too close to the axis of the implant.

Thus, the unique trajectory can be used as a tool for providing the orientation and depth of the implanted sensor unit.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A target locating and in vivo sensor system adapted for use with a therapy delivery and/or imaging source, comprising:
an external solenoid member;
a mechanism operably associated with the external solenoid member, wherein, in operation, the mechanism is configured to controllably move the solenoid external of a patient;
a controller configured to direct the movement of the mechanism, the controller being in communication with a power source configured to power the external solenoid;
at least one implantable wireless unit comprising a solenoid, wherein, in operation, the unit solenoid held internally in the patient cooperates with the external solenoid to generate a coupling signal having a signal strength that varies based on the position of the external solenoid member relative to the implanted unit; and
a computer module in communication with the controller comprising computer program code that evaluates the coupling signal strength in relation to the position of the external solenoid and determines the position of the at least one internally implanted unit.

2. A system according to claim 1, wherein the at least one implantable unit is a sensor configured to sense at least one predetermined parameter of interest in vivo, and an external reader configured to wirelessly communicate with the at least one implanted unit to obtain data associated with the at least one predetermined parameter of interest.

3. A system according to claim 1, wherein the mechanism comprises an articulated arm.

4. A system according to claim 2, wherein the external reader is configured to communicate with the implanted sensor unit using a bit encoded RF signal.

5. A system according to claim 1, wherein the at least one unit is configured to wirelessly relay an RF signal to the reader when implanted in tissue.

6. A system according to claim 1, wherein the external solenoid and the internal solenoid of the at least one sensor unit are configured to cooperate to generate a detectable coupling signal at a depth of up to at least about 14 cm.

7. A system according to claim 2, wherein the at least one sensor unit is a plurality of discrete sensor units.

8. A system according to claim 7, wherein the plurality of sensor units communicate with the external reader at the same frequency using unique bit encoded identifiers in the RF signal.

9. A system according to claim 8, wherein the plurality of sensors are configured to be individually polled by the external reader.

10. A system according to claim 1, wherein the at least one implanted sensor unit is chronically implanted for at least 1 week.

11. A system according to claim 2, wherein the at least one parameter comprises radiation dose.

12. A system according to claim 2, wherein the at least one parameter comprises temperature.

13. A system according to claim 2, wherein the controller is in communication with an external beam radiation therapy source.

14. A system according to claim 1, wherein the external solenoid and the at least one sensor unit solenoid are configured to generate a coupling signal that is detectable when the two solenoids are separated by at least about 14 cm.

15. A system according to claim 1, wherein the external solenoid generates a signal shape that varies with spatial and angular orientation with the internal solenoid of the sensor unit, and wherein said computer module comprises computer program code that receives the detected coupling signal strength and deconvolutes the signal shape with respect to position to determine the spatial location of the sensor in the subject.

16. A system according to claim 15, wherein the controller directs the mechanism to move the solenoid through a three dimensional pattern in free space to generate a corresponding response coupling signal, and wherein the computer module program code that evaluates the coupling signal strength uses the response signal generated by the three dimensional pattern to determine the position of the sensor unit.

17. A system according to claim 1, wherein the computer module further determines whether there is angular shift of the at least one sensor unit from an a priori position.

18. A system according to claim 1, wherein the articulated arm is configured to controllably move the solenoid in three dimensions.

19. A system according to claim 8, wherein the plurality of sensor units are configured to relay data regarding radiation dose and temperature to the reader.

20. A system according to claim 8, wherein the frequency is between about 100 kHz-1 MHz.

21. A system according to claim 2, wherein the computer module is configured to provide dynamic spatial data during delivery of a radiation therapy.

22. A system according to claim 21, wherein the controller and/or computer module is in communication with an external beam radiation therapy source to thereby guide and/or gate the administration of the radiation therapy during a radiation therapy session.

23. A method of obtaining spatial data and radiation dose data regarding a target in vivo treatment site, comprising:
implanting at least one sensor unit proximate and/or in a target treatment site of a patient;
sensing in vivo at least one predetermined parameter of interest using the implanted sensor unit;
wirelessly transmitting data associated with the sensed at least one parameter from the at least one sensor unit to an external reader;
providing an external coupling member located external of the patient proximate the target treatment site, the coupling member being configured to cooperate with the at least one implanted sensor to generate a coupling signal with a signal strength that varies in relation to the position of the coupling member with respect to the at least one sensor unit;
moving the coupling member;
detecting the signal strength of the coupling signal at a plurality of locations traveled based on the moving step; and
determining the position of the at least one sensor unit in the body based on the detecting step, thereby having the implanted sensor unit act as a positional marker and an in vivo sensor.

24-33. (canceled)

34. A computer program product for obtaining spatial data regarding the position of at least one implanted sensor, the computer program product comprising:
computer readable storage medium having computer readable program code embodied in said medium, said computer-readable program code comprising:
computer readable program code for determining the spatial location of a selected one of the at least one implanted sensor units using input data associated with variation in signal strength of a coupling signal generated by an external solenoid and the at least one sensor unit over different known external positions of the external solenoid.

35-40. (canceled)
